(19) United States
(12) Patent Application Publication
(10) Pub. No.: US 2026/0207478 A1
(43) Pub. Date: Jul. 23, 2026

(54) SPERM PROTECTION COMPOSITION AND LUBRICANT COMPOSITION COMPRISING THE SAME

(71) Applicant: GREENISH INC., Goyang-si, Gyeonggi-do (KR)

(72) Inventors: In Sung SUNG, Goyang-si (KR); Jun Seob SHIN, Goyang-si (KR)

(73) Assignee: GREENISH INC., Goyang-si, Gyeonggi-do (KR)

(21) Appl. No.: 19/134,878

(22) PCT Filed: Nov. 3, 2023
(86) PCT No.: PCT/KR2023/017551

§ 371 Date: Aug. 1, 2025

(30) Foreign Application Priority Data
Dec. 2, 2022 (KR) 10-2022-0166552

(51) Int. Cl. A61K 9/00; A61K 31/198; A61K 31/201; A61K 31/7004; A61K 33/04; A61K 33/42

(52) U.S. Cl. CPC A61K 9/0034; A61K 31/198; A61K 31/201; A61K 31/7004; A61K 33/04; A61K 33/42

(57) ABSTRACT

An embodiment of the present disclosure provides a composition for protecting sperm, comprising Capric acid.

### FIELD OF THE INVENTION

The present disclosure relates to a composition for protecting sperm and a lubricant composition comprising the same.

### BACKGROUND ART

Generally, subfertility or sterility is academically defined as the inability to achieve pregnancy within one year despite having normal sexual intercourse without contraception for men and women of childbearing age.

It is reported that about 15% of all couples of childbearing age experience subfertility or sterility. Conventionally, sterility was used to refer to a state where pregnancy is impossible, but recently, cases where the cause of pregnancy impairment is identified are sometimes referred to as sterility, and cases where the cause of pregnancy impairment is not identified are referred to as subfertility.

Subfertility or sterility can be caused by various factors, one of which is abnormalities in sperm.

Sperm abnormalities can be subdivided into abnormalities of the sperm itself, such as issues with sperm count, morphology, motility, volume, or pH; oligozoospermia, which is a decrease in sperm production; and asthenozoospermia, which is weak sperm activity. These problems can be partially compensated for by increasing the survival rate of sperm.

Furthermore, in industries such as animal husbandry, research on techniques for preserving sperm for species preservation and breeding is actively being conducted. However, when sperm is frozen for long-term preservation, there is a problem of decreased sperm motility or a lower survival rate upon thawing the frozen sperm.

Therefore, there is a need for research on a composition for protecting sperm that can be applied to lubricant compositions, sperm protectants for livestock animals, and compositions for protecting sperm during the thawing of frozen sperm, to increase the sperm survival rate and protect sperm for a long time.

### SUMMARY OF THE INVENTION

The present disclosure provides a solution to the above-described problems of the related art.

An aspect of the present disclosure is to provide a composition for protecting sperm with a high sperm survival rate and sperm survival maintenance rate.

Another aspect of the present disclosure is to provide a lubricant composition that is helpful for pregnancy due to its high sperm survival rate and sperm survival maintenance rate.

In accordance with one aspect of the present disclosure, a composition for protecting sperm is provided, which comprises Capric acid.

In one embodiment, the concentration of the capric acid may be 10 to 110 μg/mL.

In one embodiment, the composition for protecting sperm may further comprise one selected from the group consisting of PBS, Selenium, Ethylenediaminetetraacetic acid (EDTA), and a combination of two or more thereof.

In one embodiment, the composition for protecting sperm may further comprise PBS, 1 to 20 ng/ml of selenium, and 0.1 to 1 mM of EDTA.

In one embodiment, the composition for protecting sperm may further comprise D-fructose.

In accordance with another aspect of the present disclosure, a lubricant composition is provided, which comprises the composition for protecting sperm.

In one embodiment, the lubricant composition may further comprise a thickening agent.

The composition for protecting sperm according to an aspect of the present disclosure exhibits a high sperm survival rate and sperm survival maintenance rate, thereby showing an excellent sperm protection effect. Accordingly, it can be applied to various fields requiring a sperm protection effect, such as lubricant compositions, sperm protectants for livestock animals, and compositions for protecting sperm during the thawing of frozen sperm.

Furthermore, the lubricant composition according to another aspect of the present disclosure has a high sperm survival rate and sperm survival maintenance rate, which can be helpful for pregnancy.

The effects of the present disclosure are not limited to the effects described above, and it should be understood to include all effects that can be inferred from the configurations described in the detailed description or claims of the present disclosure.

### BRIEF DESCRIPTION OF THE DRAWINGS

FIGS. 1 to 3 are graphs showing the results of the E-N staining experiment for the Examples of the present disclosure as a sperm survival rate compared to the Comparative Example (FIG. 1: after 30 minutes, FIG. 2: after 2 hours, FIG. 3: after 4 hours).

FIGS. 4 and 5 are graphs showing the results of the HOS test for the Examples of the present disclosure as a sperm survival rate compared to the Comparative Example (FIG. 4: after 30 minutes, FIG. 5: after 2 hours).

### DETAILED DESCRIPTION

Hereinafter, aspects of the present disclosure will be described with reference to the accompanying drawings. However, the embodiments of the present disclosure may be embodied in many different forms and should not be construed as limited to the embodiments set forth herein. In the drawings, parts unrelated to the description are omitted to clearly explain aspects of the present disclosure, and similar reference numerals are assigned to similar parts throughout the present disclosure.

Throughout the present disclosure, when a part is referred to as being “connected” to another part, this includes not only cases of being “directly connected” but also cases of being “indirectly connected” with other elements interposed therebetween. In addition, when a part is said to “comprise” or “include” a certain component, it means that it can further include other components, rather than excluding them, unless there is a specific description to the contrary.

In the present disclosure, when a range of numerical values is described, the value has the precision of the significant figures provided according to the standard rules in chemistry for significant figures, unless its specific range is otherwise described. For example, 10 includes the range from 5.0 to 14.9, and the number 10.0 includes the range from 9.50 to 10.49.

Hereinafter, an embodiment of the present disclosure will be described in detail with reference to the attached drawings.

### Composition for Protecting Sperm

The composition for protecting sperm according to one aspect of the present disclosure comprises capric acid.

The capric acid is a saturated fatty acid containing 10 carbon atoms, naturally found in coconut oil, palm kernel oil, etc., with the chemical formula CH₃(CH₂)₈COOH.

The sperm may be the sperm of a mammal, but is not limited thereto. The mammal may be a human, a non-human primate, a cow, a horse, a pig, a sheep, a goat, a donkey, a rabbit, a mouse, or a cat, but is not limited thereto.

The composition for protecting sperm may exhibit an excellent sperm protection effect due to a high sperm survival rate and sperm survival maintenance rate.

The composition for protecting sperm can be applied to various fields requiring a sperm protection effect, such as lubricant compositions, sperm protectants for livestock animals, and compositions for protecting sperm during the thawing of frozen sperm.

The concentration of the capric acid may be from 10 to 110 ug/mL. For example, it can be 10 ug/mL, 15 ug/mL, 20 ug/mL, 25 ug/mL, 30 ug/mL, 35 ug/mL, 40 ug/mL, 45 ug/mL, 50 ug/mL, 55 ug/mL, 60 ug/mL, 65 ug/mL, 70 ug/mL, 75 ug/mL, 80 ug/mL, 85 ug/mL, 90 ug/mL, 95 ug/mL, 100 ug/mL, 105 ug/mL, 110 ug/mL, or a value between any two of these values. If the concentration of capric acid is less than the above range, the sperm survival rate and sperm survival maintenance rate may decrease, failing to show a sperm protection effect; if it exceeds the above range, it may not dissolve sufficiently in the composition or may excessively lower the pH of the composition, thereby reducing the activity or survival rate of sperm.

The composition for protecting sperm may comprise an aqueous solvent. For example, it may comprise purified water, PBS, or a mixture thereof as a solvent, but is not limited thereto.

The composition for protecting sperm may further comprise one selected from the group consisting of PBS, Selenium, Ethylenediaminetetraacetic acid (EDTA), and a combination of two or more thereof. The PBS can function as a solvent and a pH buffering solution, the selenium can play a role in protecting sperm from reactive oxygen species, and the EDTA can play a role in protecting sperm from reactive oxygen species or regulating pH and osmotic pressure.

The concentration of the selenium may be from 1 to 20 ng/ml. For example, it can be 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, or a value between any two of these values. If the concentration of selenium exceeds this range, cytotoxicity may be induced.

The concentration of the EDTA may be from 0.1 to 1 mM. For example, it can be 0.1 mM, 0.2 mM, 0.3 mM, 0.4 mM, 0.5 mM, 0.6 mM, 0.7 mM, 0.8 mM, 0.9 mM, 1 mM, or a value between any two of these values. If the concentration of EDTA exceeds this range, the survival rate of sperm may be reduced.

The composition for protecting sperm may further comprise PBS, 1 to 20 ng/ml of selenium, and 0.1 to 1 mM of EDTA.

The composition for protecting sperm may further comprise an acid or a base. The acid may be HCl, and the base may be NaOH or KOH, but is not limited thereto. The acid or base can play a role in adjusting the pH of the composition for protecting sperm.

The pH of the composition for protecting sperm may be from 6.8 to 8.5. For example, it can be 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, or a value between any two of these values. If the pH of the composition for protecting sperm is below this range, the sperm survival rate may decrease or sperm motility and metabolism may be reduced; if it is above this range, sperm motility and survival rate may be reduced.

The composition for protecting sperm may further comprise D-fructose. The D-fructose can be used as an energy source for sperm, thereby increasing sperm activity.

The concentration of the D-fructose may be from 0.1 to 5 mg/ml. For example, it can be 0.1 mg/ml, 0.5 mg/ml, 1 mg/ml, 1.5 mg/ml, 2 mg/ml, 2.5 mg/ml, 3 mg/ml, 3.5 mg/ml, 4 mg/ml, 4.5 mg/ml, 5 mg/ml, or a value between any two of these values. If the concentration of D-fructose is below the above range, sperm activity may be reduced; if it is above this range, cytotoxicity may be induced.

### Lubricant Composition

The lubricant composition according to another aspect of the present disclosure comprises the above-described composition for protecting sperm.

The lubricant composition can be used during sexual intercourse, and its use may improve sperm survival rate and sperm survival maintenance rate, thus being helpful for pregnancy.

The lubricant composition may further comprise a thickening agent. The thickening agent can play a role in adjusting the viscosity of the lubricant composition.

The thickening agent may be HPMC (Hydroxypropyl Methylcellulose) or guar gum, but is not limited thereto.

The lubricant composition may be formulated in the form of a solution, gel, foam, cream, or soluble film, but is not limited thereto.

Hereinafter, the examples of the present disclosure will be described in more detail. However, the following experimental results only describe representative experimental results among the examples, and the scope and content of the present disclosure cannot be interpreted as being reduced or limited by the examples. Each of the effects of the various embodiments of the present disclosure not explicitly presented below will be specifically described in the corresponding parts.

### EXAMPLES AND COMPARATIVE EXAMPLES

Compositions for protecting sperm were prepared by adding capric acid, selenium, ethylenediaminetetraacetic acid (EDTA), and D-fructose (C₆H₁₂O₆) to 1×PBS (pH 7.88) according to the compositions in Table 1 below.

TABLE 1

| | Example | Example | Example | Example | Example | Example | Comparative |
|---|---|---|---|---|---|---|---|
| Classification | 1 | 2 | 3 | 4 | 5 | 6 | Example |
| Capric acid | 20 | 50 | 100 | 20 | 50 | 100 | 0 |
| (ug/ml) | | | | | | | |
| Selenium | 10 | 10 | 10 | 10 | 10 | 10 | 0 |
| (ng/ml) | | | | | | | |
| EDTA (mM) | 0.5 | 0.5 | 0.5 | 0.5 | 0.5 | 0.5 | 0 |
| D-fructose | 0 | 0 | 0 | 2.5 | 2.5 | 2.5 | 0 |
| (mg/mL) | | | | | | | |

### Experimental Example 1

After thawing cryopreserved bovine sperm, it was treated with the compositions of Examples 1 to 6 and the comparative example. After 30 minutes, 2 hours, and 4 hours, each bovine sperm sample was stained by sequentially adding eosin solution and nigrosin solution (E-N staining). The stained bovine sperm was smeared on a slide glass and covered with a cover slip, then observed under a microscope at 1,000× magnification.

Since eosin solution passes through damaged cell membranes and stains the cell interior pink, dead sperm were observed as light pink or red after E-N staining, while living sperm were observed as transparent.

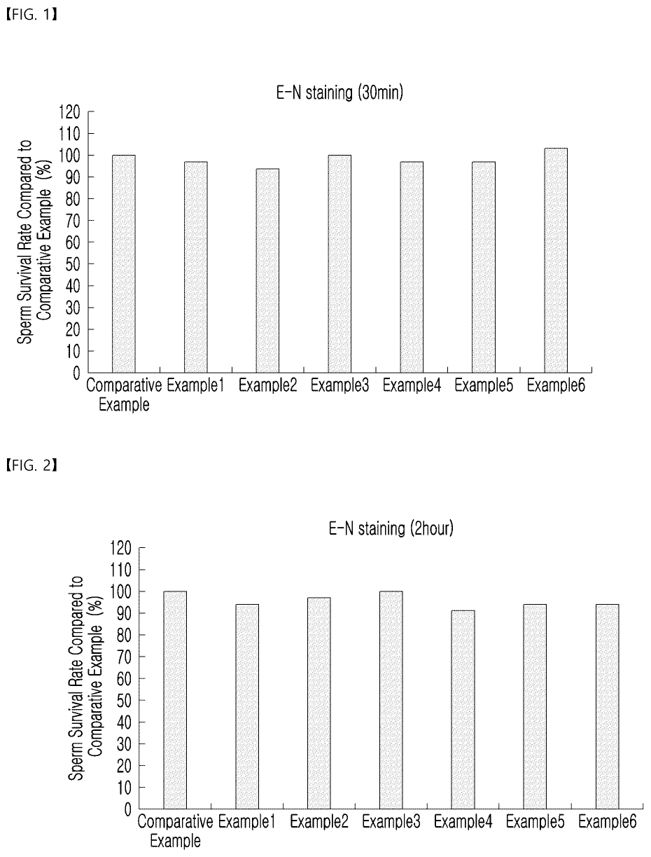

The number of living and dead sperm was counted through the microscope, and the sperm survival rate was calculated according to Equation 1 below. Then, the sperm survival rate of examples 1 to 6 relative to the Comparative Example was calculated according to equation 2 below, and the results are shown in FIGS. 1 to 3.

$$Sperm Survival Rate ( \% ) = ( Number of living sperm / Total number of sperm ) \times 100 \qquad 〈 Equation 1 〉$$

$$Sperm Survival Rate compared to Comparative Example ( \% ) = ( Sperm survival rate of Example / Sperm survival rate of Comparative Example ) \times 100 \qquad 〈 Equation 2 〉$$

Referring to FIGS. 1 to 3, it was confirmed that all the compositions of examples 1 to 6 showed a high sperm survival rate, indicating that capric acid has a significant effect on sperm protection. In particular, the composition of example 3, containing 100 ug/ml of capric acid, showed a higher sperm survival rate compared to the comparative example in the results after 30 minutes, 2 hours, and 4 hours. Example 6, which additionally contained D-fructose, also showed an excellent sperm survival rate.

Furthermore, the sperm survival rate of the compositions of examples 2 and 3 compared to the comparative example increased over time, which confirmed that their sperm survival maintenance rate was excellent.

### Experimental Example 2

After thawing cryopreserved bovine sperm, it was treated with the compositions of examples 1 to 6 and the Comparative Example. After 30 minutes and 2 hours, HOS (Hypo-Osmotic Swelling) buffer was added to each bovine sperm sample to react (HOS test). Then, each bovine sperm sample was smeared on a slide glass and covered with a cover slip, then observed under a microscope at 1,000× magnification.

The HOS test is a method that can analyze the survival rate or functional integrity of sperm through the morphological changes in the sperm tail that appear when treated with a low osmotic pressure HOS buffer. For living sperm, a phenomenon where the sperm tail curls as water permeates the sperm's plasma membrane due to osmotic pressure can be observed. Therefore, sperm observed with an uncurled tail were judged as dead, and sperm with at least a part of the tail curled were judged as living.

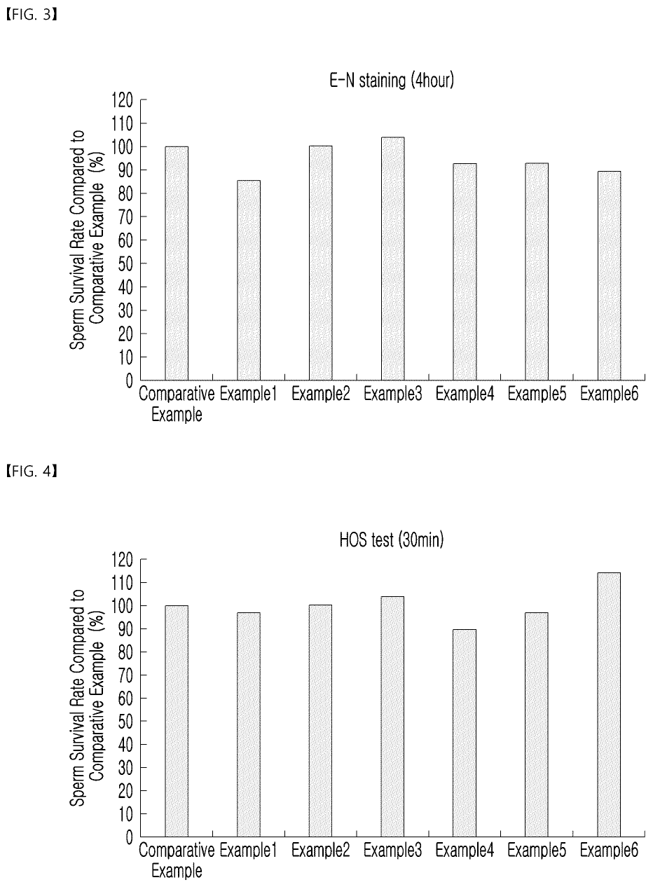

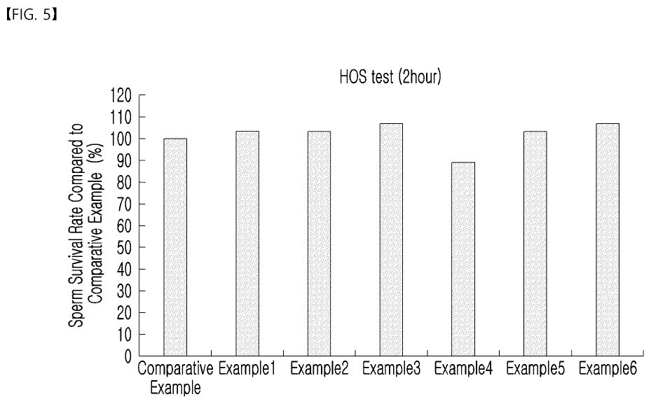

The number of living and dead sperm was counted through the microscope, and the sperm survival rate and the sperm survival rate of examples 1 to 6 relative to the Comparative Example were calculated in the same manner as in the above Experimental Example 1, and the results are shown in FIGS. 4 and 5.

Referring to FIGS. 4 and 5, it was confirmed that all the compositions of examples 1 to 6 showed a high sperm survival rate, indicating that capric acid has a significant effect on sperm protection. In particular, the compositions of examples 2, 3, and 6 showed a higher sperm survival rate compared to the Comparative Example in the results after both 30 minutes and 2 hours.

Furthermore, the sperm survival rate of the compositions of examples 1 to 3, 5, and 6 compared to the Comparative Example either increased over time or remained at a high value, which confirmed that their sperm survival maintenance rate was excellent.

### Experimental Example 3

A cytotoxicity test (24 hours) was performed on the sperm-protecting compositions of Examples 3 and 6 and the Comparative Example composition in accordance with ISO 10993-5. The results are shown in Table 2 below.

TABLE 2

| | Example | Example | Comparative |
|---|---|---|---|
| Classification | 3 | 6 | Example |
| Survival | 100 | 100 | 100 |
| rate of | | | |
| control | | | |
| group (%) | | | |
| Grade | 0 | 0 | 0 |
| Reactivity | None | None | None |

Referring to Table 2, it was confirmed that the sperm-protecting compositions of Examples 3 and 6 and the Comparative Example composition all had no cytotoxicity.

The foregoing description of the present embodiments is for illustrative purposes, and it will be understood by those of ordinary skill in the art that various changes in form and details may be made therein without changing the technical spirit or essential features described in the present disclosure. Therefore, it should be understood that the embodiments described above are illustrative in all aspects and not restrictive. For example, each component described as a single unit may be implemented in a distributed manner, and likewise, components described as distributed may also be implemented in a combined form.

The scope of the present disclosure is indicated by the claims which will be described later, and all changes or modified forms derived from the meaning and scope of the claims and their equivalent concepts should be interpreted as being included in the scope of the present disclosure.

1. A composition for protecting sperm, comprising Capric acid.

2. The composition for protecting sperm of claim 1, wherein the concentration of the capric acid is from 10 to 110 ug/mL.

3. The composition for protecting sperm of claim 1, wherein the composition further comprises one selected from the group consisting of PBS, Selenium, Ethylenediaminetetraacetic acid (EDTA), and a combination of two or more thereof.

4. The composition for protecting sperm of claim 1, wherein the composition further comprises PBS, from 1 to 20 ng/ml of selenium, and from 0.1 to 1 mM of EDTA.

5. The composition for protecting sperm of claim 1, wherein the composition further comprises D-fructose.

6. A lubricant composition, comprising the composition for protecting sperm of claim 1.

7. The lubricant composition of claim 6, wherein the lubricant composition further comprises a thickening agent.

8. A method to improve a survival or maintenance rate of sperm, comprising providing the composition according to claim 1 to a container including the sperm.
